(19) United States
(12) Patent Application Publication
(10) Pub. No.: US 2022/0273802 A1
(43) Pub. Date: Sep. 1, 2022

(54) METHODS FOR IMPROVED DELIVERY OF AMINOTHIOLS, DIMERS OF AMINOTHIOLS, AND HETERODIMERS COMPOSED OF AMINOTHIOLS

(71) Applicant: THE BURLINGTON HC RESEARCH GROUP, INC., Jericho, VT (US)

(72) Inventors: Dale M. WALKER, Jericho, VT (US); Vernon E. WALKER, Jericho, VT (US)

(21) Appl. No.: 17/574,885

(22) Filed: Jan. 13, 2022

Related U.S. Application Data
(63) Continuation of application No. 17/466,538, filed on Sep. 3, 2021, now abandoned.
Application No. 17/466,538 is a continuation of application No. 17/243,249, filed on Apr. 28, 2021, now abandoned.
Application No. 17/243,249 is a continuation of application No. 16/193,168, filed on Nov. 16, 2018, now abandoned.
Application No. 16/193,168 is a continuation of application No. 15/293,812, filed on Oct. 14, 2016, now abandoned.
Application No. 15/293,812 is a continuation of application No. 13/917,931, filed on Jun. 14, 2013, now abandoned.
(60) Provisional application No. 61/659,833, filed on Jun. 14, 2012.

(51) Int. Cl. A61K 47/54; A61K 31/145; A61K 31/661; A61K 9/00

(52) U.S. Cl. CPC A61K 47/54; A61K 31/145; A61K 31/661; A61K 47/542; A61K 9/0053

(57) ABSTRACT

The disclosure relates to methods of improving safety, efficacy, or both, of pharmaceutically active aminothiol compounds by delivering them in a thiol-protected form and, preferably intracellularly.

### CROSS-REFERENCES TO RELATED APPLICATIONS

This application is a continuation of U.S. patent application Ser. No. 17/466,538, filed Sep. 3, 2021, which is a continuation of U.S. patent application Ser. No. 17/243,249, filed Apr. 28, 2021, which is a continuation of U.S. patent application Ser. No. 16/193,168, filed Nov. 16, 2018, which is a continuation of U.S. patent application Ser. No. 15/293,812, filed Oct. 14, 2016, which is a continuation of U.S. patent application Ser. No. 13/917,931, filed Jun. 14, 2013, and claims the benefit of U.S. Provisional Appl. Ser. No. 61/659,833, filed on Jun. 14, 2012, each of which is hereby incorporated by reference in its entirety.

### BACKGROUND OF THE DISCLOSURE

The disclosure relates generally to the field of delivery of aminothiol drugs.

In the current drug delivery and metabolism systems (current drug delivery system(s)) used to achieve delivery of the phosphorothioate forms of aminothiols to cells of interest, the phosphate group serves the purpose of protecting the active metabolite from adventitious reactivity during the process of drug delivery to target and non-target cells. The phosphate group has the desirable characteristic of being removable by cell membrane-bound alkaline phosphatase. Once the parent drug has been metabolized to its active form by alkaline phosphatase, the active metabolite is taken into the cell by passive diffusion or, under some conditions, active transport by the polyamine transport system.

Problems associated with reliance upon this drug delivery and activation system adversely affect the efficacy of the phosphorothioates. For example, lymphocytes (including T-cells) only produce alkaline phosphatase during some limited duration activation phases and some developmental phases, and thus, under the majority of conditions, these cells cannot metabolize the phosphorothioates, and thus, are dependent upon distribution of the active form of the drugs from other cells that do have the ability to metabolize (dephosphorylate) these drugs.
For lymphocytes in circulation, this process is limited due to their distance from other cells, such as endothelial cells, and results in reduced drug delivery to this cell type. In addition, plasma and serum contain enzymes capable of metabolizing the phosphorothioates to their active forms where these moieties then can bind to albumen and/or be metabolized further to cytotoxic aldehydes and other derivatives. For treatment of viruses that infect lymphocytes, such as HIV
and related retroviruses, these difficulties in achieving drug delivery and
metabolism result in (i) higher drug levels in non-target cells (ii) lower drug levels in target. cells, with resultant lower therapeutic effects and higher levels of drug-induced toxicity, and/or (iii) activation of the drug in non-target sites where it is available to induce toxic effects or where it can be metabolized to toxic metabolites. New drug protecting and delivery systems are needed to overcome these problems.

### BRIEF SUMMARY OF THE SEVERAL VIEWS OF THE DRAWINGS

FIG. 1 depicts the chemical structure of the phosphorothioate compound designated amifostine.

FIG. 2 depicts the chemical structure of WR-1065, the active metabolite of amifostine

FIG. 3 depicts the chemical structure of WR-33278, the symmetrical dimer of WR-1065.

FIG. 4 depicts the chemical structure of phosphonol.

FIG. 5 depicts the chemical structure of the WR-3638, the active metabolite of phosphonol.

FIG. 6 depicts the chemical structure of the symmetrical disulfide of WR-3638.

FIG. 7 depicts the general chemical structure of the aminothiols discussed in this application. In this Figure, X can be any protecting group, including the aminothiol itself (thereby forming a homodimer), a different aminothiol (forming a heterodimer), cysteamine, a sulfur containing molecule or compound, or any other protecting group that can be removed by intracytoplasmic cellular processes.

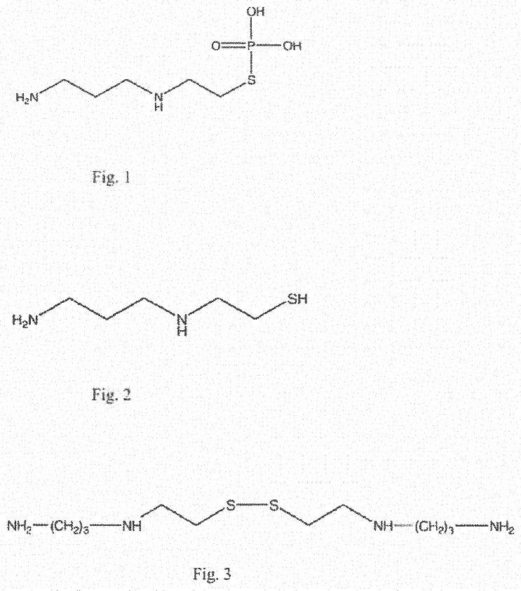

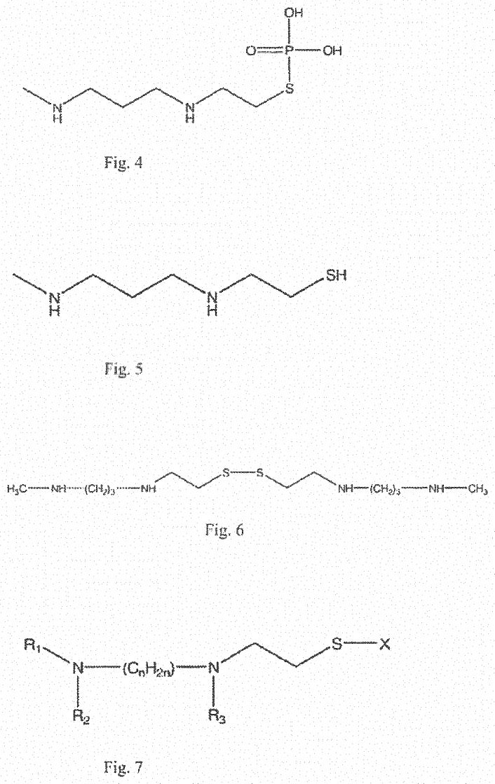

### DETAILED DESCRIPTION

The disclosure relates to improved methods of achieving intracellular delivery of aminothiol compounds.

This disclosure relates to methods for achieving improved therapeutic efficacy of aminothiols, their metabolites, analogs thereof, dimers and heterodimers of the aforementioned through the use of sulfhydryl protecting groups other than phosphate protecting groups, combined with drug delivery systems that achieve intracellular or intracytoplasmic delivery. This disclosure also relates to the use of novel, non-phosphorylated forms of the active metabolites of the phosphorothioates (aminothiols) for the purpose of achieving improved therapeutic efficacy of the drugs.

In this disclosure, metabolites of phosphorothioates include drugs described as aminothiols, heterodimers of aminothiols (also called mixed disulfides), homodimers of aminothiols (also called symmetrical dimers or symmetrical disulfides), tethered forms of the aminothiols, cysteamine, and cystamine. The aminothiols include, but are not limited to, the active metabolites of the phosphorothioates designated amifostine (WR-2721), phosphonol (WR-3689), WR-131527, structurally-related phosphorothioates, and their dephosphorylated active metabolites.

This disclosure also relates to methods for protecting the sulfhydryl moiety of these drugs during the delivery process, and then de-protecting this moiety after intracellular or intracytoplasmic delivery is achieved,

Improved sulfhydryl protecting groups combined with intracellular drug delivery system(s) for the aminothiols, their metabolites, and/or their analogs to cells where therapeutic effects are desired need to meet three conditions. First, the protecting group should have the capacity to prevent adventitious reactivity of the aminothiols during drug delivery. Second, the protecting group should be removable by systems or processes available to target cells, and third, the protecting group (e.g., following its cleavage from the aminothiol moiety) should be non-toxic to animal and human cells.

The active moieties of the phosphorothioates react readily with proteins and nucleic acids, and thus, the active forms need to be released at or near the sites where reactivity is desired as part of the therapeutic effect of the drug. Since the therapeutic effects of these drugs have been shown to occur intracellularly as opposed to extracellularly, intracellular delivery represents the optimal delivery site. Intracellular delivery will optimize opportunities for reactivity of the active drug metabolite with target cellular elements as opposed to reaction with targets that are not associated with therapeutic effects, including but not limited to extracellular targets.

Intracellular drug delivery systems that can protect aminothiols, their dimers, heterodimers, and/or analogs from adventitious reactivity during delivery, that can deliver the drug intracellularly, and that are non-toxic can be used to achieve this goal. Methods are presented below for resolving these problems by using drug formulations that do not include a phosphate group to protect the sulfhydryl group of the aminothiols. These methods then are combined with methods for intracytoplasmic drug delivery.

Intracellular delivery methods and compositions have been developed by others for effecting intracellular delivery of other drug molecules. Some of those methods and compositions (e.g., those explicitly described or referenced herein) can be used to effect intracellular delivery of aminothiols. However, it is believed that no others have previously proposed to use such compositions and methods in connection with aminothiols (in part, because no rationale for doing so is believed to have been appreciated by skilled artisans). Thus, compositions and methods that have been described by others for protecting the sulfhydryl group of an active pharmaceutical entity can be used to facilitate intracellular delivery of aminothiol compounds, even if those compositions and methods are not among those explicitly described in this disclosure.

Definitions

As used herein, each of the following ten s has the meaning associated with it in this section.

“Active moiety” is used here to refer to reactive groups such as —SH and/or —NH and the compounds bearing these groups that make up part of the structure of the active metabolites of amifostine, phosphonol, and structurally-related compounds and analogs.

“Amifostine” is the name given to the phosphorothioate form of WR-1065, WR-1065 being the biologically active moiety and physiological metabolite of amifostine.

“Drug(s)” is used here to refer to any one of the aminothiols or their structurally-related analogs, dimers, or heterodimers.

“Phosphonol” is the name given to the phosphorothioate form of WR-3789, WR-3789 being the biologically active moiety and metabolite of phosphonol.

“Phosphorothioate” is the general name given to aminothiols that have a phosphate group bound to the sulfur atom.

“WR-1065” is the name given to the active moiety of amifostine. It is used here as representative of the active moieties of phosphorothioate drugs.

### DETAILED DESCRIPTION

Amifostine, phosphonol, and structurally-related aminothiols have been shown to have therapeutic efficacy when used as chemoprotectants, cytoprotectants, radioprotectants, anti-fibrotic agents, anti-tumor agents with anti-metastatic, anti-invasive, and anti-mutagenic effects, anti-oxidants, free radical scavengers, and as anti-viral agents (Grdina 2002a,b,c, Walker et al. 2009, Poirier et al. 2009, U.S. patent application publication number 2011/0053894, and U.S. patent application publication number 2009/0239817). In these two patent application publications, experimental results showed that WR-1065, the active metabolite of amifostine, exhibits antiviral efficacy against HIV, influenza virus A and B, and three species of adenovirus. Later studies also demonstrated efficacy against. SIV (Poirier et al., 2009, AIDS Res. Ther. 6:24).

In the following discussion, amifostine and its active metabolite WR-1065 will be used as representative examples of all aminothiols, phosphorothioates, their analogs, and the active metabolites of the parent drugs.

Amifostine contains a phosphate group bound in place of the hydride group of the sulfhydryl moiety of WR-1065. The phosphate group acts to protect the sulfhydryl group from adventitious reactivity during the drug delivery process. Once the drug is in the vicinity of cells, the phosphate group must be removed by dephosphorylation in order for the drug to be active (reviewed in Grdina et al., 1995, Carcinogenesis 16:767-774).

Amifostine is metabolized to its active metabolite WR-1065 by alkaline phosphatase on the plasma membrane surface. WR-1065 produced by dephosphorylation of amifostine is taken up rapidly into cells where it can be metabolized further (Purdie et al., 1983, Int. J. Radiat. Biol. Relat. Stud. Phys. Chem. Med. 43:517-527; Shaw et al., 1996, Semin. Oncol. 23:18-22). Oxidative metabolites of WR-1065 (the active thiol) include WR-33278 (the symmetrical disulfide), WR-1065-cysteine, WR-1065-glutathione, cysteamine, and other mixed disulfides (Shaw et al., 1996, Semin. Oncol. 23:18-22). Amifostine (WR-2721) without dephosphorylation to its active metabolite WR-1065 had no radioprotective effect upon mouse L cells in culture (Mori et al., 1983, Int. J. Radiat. Biol. Relat. Stud. Phys. Chem. Med. 44:41-53). In contrast, radioprotective activity was observed for WR-2721 incubated with mouse liver homogenate (which contains an active alkaline phosphatase), which dephosphorylated WR-2721 to WR-1065 (Mori et al. 1983). Activity also was observed for WR-1065 alone, showing that WR-2721 must be dephosphorylated before it is active. Dephosphorylation of other phosphorothioates also is required to convert them into their active forms. For example, the parent drug WR-151327 is metabolized by alkaline phosphatase to its active form WR-151326 and its symmetrical disulfide WR-25595501, which also is active (Vaishnav et al., 1996, J. Pharm. Biomed. Anal. 14:317-324).

The active form of amifostine (WR-1065) must be present inside of cells for beneficial effects to be observed. WR-2721 (amifostine), WR-1065, WR-33278, WR-1065-Cys, and other disulfide forms of the parent compound WR-2721 did not show evidence of activity if present outside of V79 cells (Smoluk et at., 1988, Cancer Res. 48:3641-3647). In contrast, intracellular levels of WR-1065 correlated with significant protection against gamma-radiation (Smoluk et al. 1988). Results were similar for HeLa cells, me-180 cells, Ovary 2008 cells, HT-29/SP-1d cells, and Colo 395 tumor cell lines (Smoluk et al. 1988). For optimal cytoprotection, sufficient and sustained intracellular levels of WR-1065, the active form of amifostine, were necessary (Souid et al., 1998, Cancer Chemother. Pharmacol. 42:400-406). If the cells were transferred to drug-free medium for 4 hours before exposure to radiation, the intracellular levels of WR-1065 and WR-33278 decreased markedly along with cytoprotection from radiation damage (Grdina et al. 1995). In vivo tissue levels of WR-1065 were similar in monkeys and in humans and tissue levels of drug were informative for cytoprotective effects (Cassatt et al., 2002, Semin. Radiat. Oncol. 12:97-102).

The sulfhydryl moiety of amifostine is involved in its therapeutic effects (Grdina et al., 2000, Drug Metabol. Drug Interact. 16:237-279; Grdina et al., 2002a, Semin. Radiat. Oncol. 12:103-111; Grdina et al., 2002b, Mil. Med. 167:51-53; Grdina et al., 2002c, Radiat. Res. 163:704-705), and is protected from adventitious reactively during drug delivery by the addition of a phosphate group, resulting in the phosphorothioate form of the drug. The phosphate group is removed when the drug is brought into close proximity to cell plasma membranes and/or the drug is taken up into the plasma membrane. The dephosphorylation step is carried out by membrane-bound alkaline phosphatase, an enzyme that is produced by many, but not all human and animal cells. After removal of the phosphate group, the active moiety is taken up into the intracellular milieu from which it can be distributed further to subcellular organelles or to other cells, and where therapeutic effects are induced. Cellular uptake of many, but not all forms of the aminothiols occurs by passive diffusion, but some drug forms are taken up by active transport through the polyamine transport system, and active transport of other drug forms may occur at some drug concentrations but not others (Grdina et al. 2000, 2002a,b,c). For cells that cannot take up the drug and/or cannot metabolize the drug, the active form is delivered to these cells via cell- and tissue-distribution processes.

Previously known methods for administering phosphorothioates to a human or animal include, but are not limited to, oral delivery, intraperitoneal injection, subcutaneous injection, intravenous injection, inhalation, incorporation into nanoparticles (Pamujula et al., 2004a, J. Pharm. Pharmacol. 56:1119-1125; Pamujula et al., 2004b, Eur. J. Pharm. Biopharm. 57:213-218; Pamujula et al., 2005, Int. J. Radiat. Biol. 81:251-257), or using other drug delivery systems.

Plasma-membrane bound alkaline phosphatase is a GPI-anchored protein (Marty et al., 1993, Immunol. Lett. 38:87-95) that is expressed by some, but not all, cell types. Alkaline phosphatase also is present intracellularly in the rough endoplasmic reticulum where it is synthesized, in the Golgi apparatus where additional processing may occur, in Golgi-derived vesicles, in some lysosomes, and around the nuclear envelope (Tokumitsu et al., 1983, J. Histochem. Cytochem. 31:647-655). Its localization varies with cell cycle in B lymphocytes (Souvannavong et al., 1994, J. Leukoc. Biol. 55:626-632), with synthesis occurring around the mitotic phase of the cell cycle (Tokumitsu et al., 1981, J. Histochem. Cytochem. 29:1080-1087). Plasma membrane-bound alkaline phosphatase is dependent upon correct microtubule organization to achieve its correct orientation in the cell membrane (Gilbert et al., 1991, J. Cell Biol. 113:275-288). B lymphocytes can shed alkaline phosphatase into the surrounding cellular milieu (Burg et al., 1989, J. Immunol. 142:381-387) and alkaline phosphatase also is present in serum.

Importantly, alkaline phosphatase expression is not uniform across all cell types or across all cell states or conditions, but instead is highly variable. The net effect of this variation is that reliance upon cell membrane-bound alkaline phosphatase as the mechanism for activation of a parent drug to its active form is unreliable at best, and not functional in many cases. In addition, amifostine can be cytotoxic. At least some of its toxicity is related to the fact that it can be metabolized further to aldehydes and to hydrogen peroxide, WR-1065 (possibly also amifostine) circulating in the blood stream is metabolized further by copper-dependent amine oxidases to the aforementioned toxins. These metabolites are both directly cytotoxic to cells and indirectly toxic through induction of oxidative stress, a condition that can lead. to increased cell death, cell damage, mitochondrial damage, and/or aberrant cell function. Thus, failure to metabolize amifostine in the desired cellular or organ milieu can have deleterious effects.

The above considerations have important implications for the use of amifostine and the phosphorothioates, including but not limited to their use as an antiviral agents. Many kinds of injuries and essentially all infectious agents including bacteria, viruses, and parasites, induce expression of inflammatory stresses in affected cells and organ systems. This leads to changes in the expression of alkaline phosphatase, release of alkaline phosphatase into the intercellular and/or extracellular milieu, and/or changes in circulating levels of the enzyme. These events will result in metabolism of amifostine in tissue compartments where it has no effect, or to metabolism in areas where its active moiety WR-1065 is metabolized readily to toxic bi-products, thereby increasing the overall toxicity of the drug without a concomitant therapeutic effect. For target cells that cannot metabolize amifostine because they express little to no plasma membrane bound alkaline phosphatase, in vivo antiviral effects are limited due to the inability of these cells to metabolize amifostine to its active form.

Taken together, these considerations show that reliance upon the current drug delivery system introduces several significant problems that are presented below. In brief, these problems include (1) inability to metabolize the drug to its active form by some cell types, (2) inability to activate/metabolize the drug under some physiological or disease conditions, (3) activation of the drug in milieus where its activity is not desired, (4) activation of the drug at a distance from the optimal cellular or subcellular milieu, and (5) lack of ability to achieve targeted cell delivery or targeted cell exclusion.

First, some cells to which drug delivery is desired do not produce membrane-bound alkaline phosphatase, or produce it only under limited conditions, or only produce it during developmental stages that are of limited duration. As an example, lymphocytes, including T-cells, are a cell type that is highly sensitive to infection by the HIV virus. For cell types that do not produce alkaline phosphatase, tissue distribution from cells that initially metabolized and took up the drug, such as endothelial cells, is necessary for drug delivery. This process is inefficient at best, and can be altered by disease states such as infection, inflammation, or other conditions.

Second, in some disease states, such as during inflammation or infection, membrane-bound alkaline phosphatase expression and localization are altered. Alkaline phosphatase is released into the extracellular milieu during some infectious conditions as a generalized response to pathogens. Extracellular production of alkaline phosphatase is sufficiently pronounced in cases of HIV/AIDS that one group of investigators has proposed that circulating blood levels of alkaline phosphatase levels can be used as a diagnostic marker of the disease (Murthy et al., 1994, Arch. Pathol. Lab. Med. 118:873-87). Release of alkaline phosphatase into the extracellular milieu can result in metabolism of phosphorothioates to their active metabolites at a distance from cell membranes. This reduces uptake by cells, increases the availability of metabolites for participation in non-therapeutic reactions, and makes the active moieties available for further metabolism to aldehydes and other compounds with cytotoxic effects.

Third, extracellular, non-membrane-bound alkaline phosphatase levels are high in some extracellular spaces such as in the human intestinal lumen and in mucus secretions in the human lung. The presence of the enzyme in these sites results in metabolism of the drug to its active form at a distance from cell membranes, thereby adversely affecting drug delivery from these spaces into cells. This is problematic since delivery of the drug to cells of the intestine or the lung is desirable to achieve some drug-related effects such as anti-viral therapy.

Finally, reliance upon the current drug delivery systems results in activation of the drug outside the plasma membrane of cells, and upon subsequent passive uptake for delivery into the cell cytoplasm where therapeutic effects occur. Passive and active cellular drug uptake processes can be affected by disease-associated stress conditions such as inflammation or injury, with the result that drug uptake is reduced or blocked completely. Additional problems stem from the fact that the current drug delivery system does not allow for targeted drug delivery or targeted cell exclusion. This problem is especially important since studies show that there are significant cell type and tissue type differences in tolerance to intracytoplasmic concentrations of WR-1065 (Walker et al., 2009, Environ. Mol. Mutagen. 50:460-472), and that this tolerance varies with disease states (Poirier et al. 2009). Thus, the ability to target specific cell types for enhanced drug delivery, and/or to exclude other cell types from significant drug delivery is expected to have a significant impact upon overall drug efficacy. Drug delivery systems with these capabilities also are expected to reduce drug toxicity by lowering the amount of drug available for conversion to toxic metabolites.

Taken together, these findings support the conclusion that reliance upon a phosphate group for protection of the sulfhydryl moiety of an aminothiol during delivery, and reliance upon alkaline phosphatase for metabolism of the parent drug to its active moiety have significant disadvantages that can affect drug efficacy adversely. The above considerations demonstrate the need for new drug formulations and/or new drug delivery system(s). Methods for achieving these results are presented below.

General Considerations

Three criteria should be satisfied to address the above described problems. Sulfhydryl groups are highly reactive moieties that will form covalent bonds with a variety of moieties present in the bodies and cells of living organisms. Thus, therapeutic drugs that contain one or more sulfhydryl groups that are known or hypothesized to have roles in the pharmacological effects of those drugs require protection of the sulfhydryl moiety during delivery to prevent reactivity with neighboring molecules not related to the drug's desired therapeutic effects. To achieve this protection, any molecular group can be used if it meets the requirements that (i) it achieves the desired protective effect during delivery, (ii) it can be removed intracellularly, and (iii) it is not toxic to cells (either before or after removal from the active aminothiol moiety).

Any method that achieves intracellular drug delivery, including but not limited to delivery into intracellular organelles, will serve the purpose of delivering aminothiol drugs to a milieu where its activity is desired and where it will have a beneficial effect. That is, the observations made in this disclosure relate importantly to realization that intracellular delivery of an intracellularly-cleavable aminothiol/thiol-protecting-moiety conjugate beneficially affects administration of aminothiols. The observations made in this disclosure also relate to realization that intracellular delivery—however achieved—of an aminothiol compound having a reactive active moiety is advantageous relative to extracellular delivery of the corresponding phosphorothioate of the aminothiol compound.

Targeted cell delivery and/or targeted cell exclusion is desirable because of the recognized toxicity of aminothiols. For delivery by certain methods, such as oral delivery or inhalation delivery, the delivery method or system should be one that has the capacity to protect the drug from degradation by, and/or reactivity with, enzymes found in the lumen of organs through which the drug will pass. Thus, for oral delivery the methods must achieve protection from luminal enzymes and factors of the gastrointestinal tract, and for inhalation delivery, the methods must protect against degradation by lung exudates/secretions.

Finally, to achieve drug activation, any group used to protect the sulfhydryl group of WR-1065 must be one that can be released or removed once the drug has been successfully delivered into the cytoplasm of target and/or non-target cells.

Methods to Protect the Active Form of the Drug During Delivery and Release it Once Delivery has Been Completed

In general, any compositions or method(s) that provide protection of the sulfhydryl group of the aminothiols during delivery and that also result in release of the active form of the drug following delivery to the desired site(s) can be used. Because protection systems should have the characteristic of being able to release the active moiety of the drug once intracytoplasmic delivery has been achieved, systems that address both protection during delivery and release after delivery are discussed together and are presented below as items (i)-(v).

(i) Protect the active form of the aminothiol during drug delivery through use of the homodimers of the aminothiols, which are bound through their sulfur atoms. As an example, WR-33278 (the symmetrical disulfide of WR-1065) can be delivered instead of amifostine. The dimer's disulfide bond provides protection to the sulfhydryl groups and this bond is reducible through redox reactions that occur in the reducing environment of the cell cytoplasm to yield two molecules of WR-1065 (this process is a type of bioreductive activation (Gharat et al., 2001, Int. J. Pharm. 219:1-10)). In addition, the homodimers (symmetrical dimers) of aminothiols also have activity similar to that of their reduced forms. In cases where cellular redox status and/or reactions can be perturbed, as can occur in some diseases states and/or under some types of stress conditions, one or more molecules of a reducing agent can be incorporated into the delivery system or composition, along with the symmetrical dimer of the aminothiol. Another method to enhance reduction-associated intracellular processes is to include other therapies to improve or restore cellular redox status, such as anti-oxidant therapy, during therapy with the symmetrical dimer.

(ii) Protect the active form of the aminothiol during drug delivery through use of heterodimers of the aminothiols. Aminothiol A can be synthesized bound through its sulfur atom to the sulfur atom of aminothiol B, thereby forming a heterodimer. The disulfide bonds are susceptible to reduction by cellular redox reactions as described above. Note that both aminothiols so bound can have the same desired effects upon cells and/or pathogens, albeit with differing degrees of efficacy.

(iii) Protect the active form of the aminothiol by tethering it or binding it to a moiety from which it can dissociate within the intracytoplasmic milieu. It should be noted that this method is similar to the one above, but involves binding the aminothiol to non-aminothiol molecules (which may or may not have pharmaceutical activities). Potential moieties include peptides, cell penetrating peptides, sulfur-containing amino acids, glutathione, sulfur- or thiol-containing anti-oxidants, or other thiol- or sulfur-containing non-protein molecules, including but not limited to cysteamine. In some cases, the drug delivery method can have an effect similar to that of the aminothiol that is being delivered. For example, polyunsaturated liposomes, which can be used as a drug delivery system, have antiviral activity against hepatitis B and C viruses and against HIV (Pollock et al., 2010, Proc. Natl. Acad. Sci. U.S.A. 107:17176-17181).

(iv) Deliver the aminothiol of interest immobilized or locked in a matrix so that it cannot react until released into the cytoplasm of target cells.

(v) Other methods for protection of the sulfur moiety of the aminothiols include (a) using a photoreversible thiol tag, (b) using S-cysteinylation, (c) using Trityl (Trityl has been used in the molecule C₃₁H₃₇NO₄S ((R)-tert-butyl-2-[(tert-butoxycarbonyl)amino]-3-(tritylsulfanyl)propanoate)) to protect the sulfhydryl group (Koziol et al., 2001, Chem. Pharm. Bull. (Tokyo) 49:418-423).

In addition to the above, a variety of methods have been described for protection of thiol groups and/or for drug delivery, including the following items (A)-(E).

(A) Delivery systems using biodegradable bonds, such as those described in Kim et al., 2010, J. Biomed. Sci. 17:61.

(B) Cysteine-based cell penetrating peptide drug delivery system, such as those described in Jha et al. 2011.

(C) Reducible disulfide bonds, such as those described in Cohen et al., 2012, Biomaterials 33:614-623; Herlambang et al., 2011, J. Control. Release 155:449-457; Li et al., 2011, Biomaterials 32:6633-6645; Liu et al., 2011, Biomacromolecules 12:1567-1577; Nguyen et al., 2011, Biomed. Mater. 6:055004; Park et al., 2010, Small 6:1430-1441; Rahbek et al., 2010, J. Drug Target, 18:812-820; Zhang et al., 2010, J. Control. Release 143:359-366; Zhang et al., 2011b, Biomaterials 32:4604-4608; Zhao et al., 2011, Biomaterials 32:5223-5230.

(D) Gold-based protective and delivery systems, such as those described in Pissuwan et al., 2011, J. Control. Release 149:65-71.

(E) Stabilization in aqueous solution, such as systems described in Bawa et al., 2011, Nanomedicine 8:647-654.

Methods for Intracellular/Intracytoplasmic Drug Delivery to Target and Non-Target Cells

In general, any method described in the literature or developed in the future that results in intracytoplasmic delivery of the aminothiols for the purpose of achieving therapeutic effects can be used. Targeted drug delivery and targeted drug exclusion are desirable but not necessary.

A variety of particulate carriers for intracellular drug delivery have been developed and/or described. Nanoparticles also are referred to as nanovesicles, nanocarriers, or nanocapsules and include lysosomes, micelles, capsules, polymersomes, nanogels, dendritic and macromolecular drug conjugates, and nano-sized nucleic acid complexes. A summary of categories into which nanoparticles are sometimes divided includes the following items (1)-(18),

(1) Cell penetrating agents such as amphiphilic polyproline helix P11LRR, such as those described in Li et al., 2010, J. Control. Release 142:259-266 or peptide-functionalized quantum dots, such as those described in Liu et al., 2010, J. Nanosci. Nanotechnol. 10:7897-7905.

(2) Carriers responsive to pH, such as carbonate apatite (Hossain et al., 2010, J. Control. Release 147:101-108).

(3) C2-streptavidin delivery systems, which have been used to facilitate drug delivery to macrophages and T-leukemia cells, such as those described in Fahrer et al., 2010, Biol. Chem. 391:1315-1325.

(4) CH(3)-TDDS drug delivery systems.

(5) Hydrophobic bioactive carriers, such as those described in Imbuluzqueta et al., 2011, Acta Biomater. 7:1599-1608.

(6) Exosomes, such as those described in Lakhal et al., 2011, Mol. Ther. 19:1754-1756; Zhang et al., 2011a, Drug Discov. Today 16:140-146.

(7) Lipid-based delivery systems, such as those described in Kapoor et al., 2012, Int. J. Pharm. 427:35-57; Bildstein et al., 2010, J. Control. Release 147:163-170; Foged, 2012, Curr. Top. Med. Chem. 12:97-107; and Holpuch et al., 2010, Pharm. Res. 27:1224-1236, including microtubules, such as those described in Kolachala et al., 2011, Laryngoscope 121:1237-1243.

(8) Liposome or liposome-based delivery systems.

(9) Micelles, including disulfide cross-linked micelles, such as those described in Li et al. 2011. Carriers with disulfide bonds can be formulated so that one or more disulfide bonds link to the aminothiol. A variety of micelles have been described, such as phospholipid-polyaspartamide micelles for pulmonary delivery.

(10) Microparticles, such as those described in Ateh et al., 2011, Biomaterials 32:8538-8547.

(11) Molecular carriers, such as those described in Hettiarachchi et al., 2010, PLoS One 5:e10514.

(12) Nanoparticles referred to as ‘nanocarriers’, such as those described in Gu et al., 2011, Chem. Soc. Rev. 40:3638-3655 some of which have been formulated for delivery of agents to HIV infected cells, such as those described in Gunaseelan et al., 2010, Adv. Drug Deliv. Rev. 62:518-531.

(13) Nanoscopic multi-variant carriers.

(14) Nanogels, such as those described in Zhan et al., 2011, Biomacromolecules 12:3612-3620 and Zhang et al. 2010.

(15) Hybrid nanocarrier systems, which consist of components of two or more particulate delivery systems, such as those described in Pittella et al., 2011, Biomaterials 32:3106-3114. Copolymeric micelle nanocarrier, such as those described in Chen et al., 2011, Biomacromolecules 12:3601-3611; liposomal nanocarriers, such as those described in Kang et al., 2011, J. Drug Target 19:497-505.

(16) Nanoparticles can be constructed with a variety of nanomaterials, such as those described in Al-Jamal et al., 2010, FASEB J. 24:4354-4365; Adeli et al., 2011, Nanomedicine 7:806-817; Bulut et al., 2011, Biomacromolecules 12:3007-3014.

(17) Peptide-based drug delivery systems, which include a variety of cell penetrating peptides and including but not limited to TAT-based delivery systems, such as those described in Johnson et al., 2011, Methods Mol. Biol. 683:535-551.

(18) Polymers or copolymer-based delivery systems, such as those described in Edinger et al., 2011, Wiley Interdiscip. Rev. Nanomed. Nanobiotechnol. 3:33-46.

Additional intracellular drug delivery systems that may be considered to fall into the category of nanoparticles include the following items (a)-(u).

(a) Aptamers, such as those described in Orava et al., 2010, Biochim. Biophys. Acta 1798:2190-2200.

(b) Bacterial drug delivery systems, such as those described in Pontes et al., 2011, Protein Expr. Purif. 79:165-175.

(c) Protein-based, self-assembling intracellular bacterial organelles (bacterial shells), such as those described in Corchero et al., 2011, Microb. Cell. Fact. 10:92.

(d) Blended systems, such as those described in Lee et al., 2010, Mol. Biosyst. 6:2049-2055.

(e) Covalently modified proteins, such as those described in Muller, 2011, Curr. Issues Mol. Biol. 13:13-24.

(f) Drug-loaded irradiated tumor cells, such as those described in Kim et al. 2010.

(g) Dual loading using micellplexes, such as those described in Yu et al., 2011, ACS Nano 5:9246-9255.

(h) Ethosomes, such as those described in Godin et al., 2003, Crit. Rev. Drug Carrier Syst. 20:63-102.

(i) Inhalation-based delivery systems, such as those described in Patton et al., 2010, J. Aerosol Med. Pulm. Drug Deliv. 23 Suppl. 2:S71-87.

(j) Irradiated tumor cell-based delivery system, such as those described in Kim et al. 2010.

(k) Lipid-based carriers.

(l) Lipospheres, such as acoustically active lipospheres.

(m) Microencapsulated drug delivery, such as those described in Oettinger et al., 2012, J. Microencapsul. 29(5):455-462 or Pavlov et al., 2011, Macromol. Biosci. 11:848-854.

(n) A delivery system referred to as molecular umbrellas, such as those described in Cline et al., 2011, Bioconjug. Chem. 22:2210-2216.

(o) Niosomes (non-ionic surfactant-based liposomes).

(p) Photo-activatible drug delivery systems.

(q) Polymeric microcapsule, such as those described in Pavlov et al., 2011.

(r) Self-emulsifying drug delivery system, such as those described in Lei et al., 2010, Mol. Pharm. 7:844-853.

(s) Trojan horse delivery systems.

(t) Vesicles including but not limited to reduction sensitive vesicles, such as those described in Park et al. 2010.

(u) Viral vectors and viral-like systems, such as those described in Bacman et al., 2010, Gene Ther, 17:713-720 or Chailertvanitkul et al., 2010, Curr. Opin. Biotechnol. 211:627-632).

It should be noted that the above listed drug delivery systems can be used in combination with each other. They also can be engineered further to provide target cell or tissue type delivery or targeted cell/tissue-type exclusion. In addition, new nanoscopic delivery systems are being developed frequently, and a variety of materials for use in the formation of nanoscopic drug delivery vehicles is expanding rapidly.

The above delivery systems can be used in combination with enhanced delivery techniques. Examples of such techniques include the following items (I)-(XIV).

(I) Amphotercin B-mediated drug delivery enhancement.

(II) Ultrasound-mediated techniques, such as those described in Grimaldi et al., 2011, Spectrochim. Acta A Mol. Biomol. Spectrosc. 84:74-85 or Yudina et al., 2011, J. Control. Release 155:442-448.

(III) Temperature-sensitive delivery and/or release systems.

(IV) pH-sensitive delivery and/or release systems.

(V) Redox-responsive delivery systems, such as those described in Zhao et al. 2011.

(VI) Bioreducible delivery systems, such as those described in Liu et al. 2011.

(VII) Methods to enhance endo-lysomal escape, such as those described in Paillard et al., 2010, Biomaterials 31:7542-7554.

(VIII) Inhalation methods, such as those described in Zhuang et al., 2011, Mol. Ther. 19:1769-1779.

(IX) Methods to enhance oral delivery, such as those described in Muller 2011.

(X) Targeted cell delivery systems, some of which have been developed for use in the delivery of anti-HIV drugs, such as those described in Gunaseelan et al. 2010; Kelly et al., 2011, J. Drug Deliv. 2011:727241; and Bronshtein et al., 2011, J. Control. Release 151:139-148).

(XI) Slow or on-demand release systems, such as those described in Hu et al., 2012, ACS Nano 6:2558-2565.

(XII) Targeted delivery to one or more receptors, such as those described in Ming, 2011, Expert Opin. Drug Deliv. 8:435-449.

(XIII) Targeted delivery to one or more different subcellular organelles, such as those described in; Paulo et al., 2011, Nanotechnology 22:494002; and Zhang et al. 2011.

(XIV) Methods to improve or to regulate drug uptake, such as those described in Ma et al., 2011, Int. J. Pharm. 419:200-208 or Lorenz et al., 2010, Macromol. Biosci. 10:1034-1042.

It should be noted that delivery of amifostine, the phosphorothioate, using nanoparticles has been reported previously (Pamuljuma et al. 2004, 2004, 2005). Pamuljuma and colleagues hypothesized that once the phosphorothioate-containing nanoparticle is delivered into cells, the phosphorothioate is released and it enters the cell membrane where it is metabolized to its active form by alkaline phosphatase. The active metabolite is released outside the cell and then is taken back up through passive or active diffusion. For the reasons presented above, this delivery system does not resolve the problems associated with dependence upon alkaline phosphatase for drug activation, and also fails to address the potential toxicity problems associated with activation of the drug outside of cells.

Methods for Release of the Active Form of the Drug Intracytoplasmically to Achieve Therapeutic Effects

In general any drug delivery system and/or drug protection method that includes the capacity to release the active form of the drug following intracytoplasmic delivery can be used. The key to the selection of one or more of the protection and delivery systems described above is to recognize that once the drug has been delivered into the cytoplasm of target cells, it should be released in its active form.

Methods for Drug Administration to Humans or Animals

The above improved drug delivery systems can be administered using any appropriate drug administration method(s) known or described in the future, including but not limited to intravenously, subcutaneously, orally, intraperitoneally, and/or by transdermal patch.

### EXAMPLES

The subject matter of this disclosure is now described with reference to the following Examples. These Examples are provided for the purpose of illustration only, and the subject matter is not limited to these Examples, but rather encompasses all variations which are evident as a result of the teaching provided herein.

In the examples below, an intracytoplasmic drug delivery system of the type described above, which provides protection from adventitious reactivity during delivery and results in release of the active from of the drug after delivery, is referred to as ‘the improved drug delivery system’.

### Example 1

Example of the Use of the Improved Drug Delivery System to Achieve Improved Cytoprotection, Including Radioprotection, Chemoprotection, Anti-Oxidant Effects, Free Radical Scavenging, and/or Cytoprotection from an Aminothiol

Animals or humans exposed to radiation, chemicals, chemotherapeutic agents, toxic therapeutic drugs such as nucleoside analogs, or toxic agents or conditions can benefit from treatment with cytoprotective drugs. Oral administration of an aminothiol using an intracytoplasmic drug delivery system as described above will result in improved overall cytoprotection of the affected animal or human. Use of the above drug delivery system will result in incorporation of the drug into the cytoplasm of cells of the gastrointestinal system while avoiding activation of the drug in the intestinal lumen where no therapeutic effects have been described. Thus, oral delivery will become useful for the aminothiols, something that is not currently practical.

Cells of the gastrointestinal system are among the most sensitive to cytotoxic conditions, and protection of this cell type will result in improved overall health and survival of the organism through retention of the ability to absorb fluids and nutrients. Distribution to cells outside the gastrointestinal system through the use of modifications to achieve targeted drug delivery will result in delivery of the drug beyond the cells of the GI tract, so that widespread cytoprotection will be obtained. Another method of achieving a similar effect will be to deliver the drug via several different avenues simultaneously, such as orally and subcutaneously.

This use resolves a problem with aminothiol-induced cell cytotoxicity. Walker et al. (2009) found that there were large differences in the levels of aminothiols that were toxic to cells. Data reported by Poirier et al. (2009) showed that aminothiol cytotoxic effects also varied depending upon the disease state of the cells. Taken together, these findings support the conclusion that improved overall cytoprotection of a variety of differing cells types being exposed to a toxic agent or condition is achieved by obtaining differing levels of the therapeutic aminothiol in cells, depending upon the individual tolerances of those cells for the aminothiol. This difference in tolerance can be as much as 100-fold or greater. The improved drug delivery system will make it possible to achieve varied intracytoplasmic aminothiol drug concentrations within a range of target cells so that the optimal cytoprotective effects for the whole organism will be achieved.

### Example 2

Example of the Use of the Improved Drug Delivery System to Achieve Improved Antiviral Effects

Viruses that infect humans and animals usually have a few target cell types that they infect and in which they replicate. The improved drug delivery system has the ability to enhance delivery to cell types of interest, including to cells to which aminothiol delivery is difficult using other delivery systems.

For HIV infection, it is critical to control drug replication in T lymphocytes and in circulating monocytes. The virus appears to infect and to reside in other cell types as well, but control of viral infectivity and replication in cells other the T cells and monocytes is not sufficient to control the viral infection. These types of circulating cells are particularly difficult to target for drug delivery using drug delivery systems other than the improved drug delivery system for reasons described above.

Using the improved drug delivery system, active drug is delivered directly into the cytoplasm of T cells, other lymphocytes, monocytes, and other cell types infected by the virus, where replication and completion of the viral life cycle takes place. Because lymphocytes are circulating cells, drug delivery can take place in the circulation. Methods to obtain slow or prolonged delivery can be incorporated into the improved drug delivery system, so that more uniform or sustained intracytoplasmic drug concentrations can be achieved. Cell type-specific intracytoplasmic drug concentrations can be achieved using cell targeting methods and variations upon drug administration methods as described above. Taken together, application of these methods will improve drug therapeutic effects by achieving the optimal drug concentration in cell types of interest while limiting the availability of drug for further metabolism to toxic metabolites.

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

What is claimed is:

1. A method of administering an aminothiol compound to a subject in need of aminothiol therapy, the method comprising administering the aminothiol in a thiol-protected form.

2. The method of claim 1, wherein the thiol-protected form of the aminothiol comprises the aminothiol conjugated with an intracellularly-cleavable thiol protecting group.

3. The method of claim 1, wherein the intracellularly-cleavable protecting group is selected from the group consisting of a peptide, a sulfur-containing amino acid, glutathione, a sulfur-containing antioxidant, an oxygen-containing antioxidant, a photoreversible thiol tag, and (R)-tert-butyl-2-[(tert-butoxycarbonyl)amino]-3-(tryitylsulfanyl)propanoate.

4. The method of claim 1, wherein the thiol-protected form of the aminothiol is selected from the group consisting of a homodimer of the aminothiol, a heterodimer of the aminothiol and a different aminothiol, and cysteamine.

5. The method of claim 1, wherein the thiol-protected form of the aminothiol has the structure 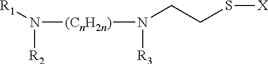
wherein X is a an intracellularly-cleavable protecting group;
wherein each of R₁, R₂, and R₃ is independently selected from hydrogen and C₁₋₆ alkyl, and
wherein n is an integer of from 1 to 10.

6. The method of claim 1, wherein the thiol-protected form of the aminothiol is administered in an intracellular delivery system.

7. The method of claim 6, wherein the intracellular delivery system is selected from the group consisting of: (a) systems comprising a cell penetrating agent, (b) pH-responsive carriers, (c) C2-streptavidin delivery systems, (d) CH(3)-TDDS drug delivery systems, (e) hydrophobic bioactive carriers, (f) exosomes, (g) lipid-based delivery systems, (h) Liposome-based delivery systems, (i) micellar delivery systems, (j) microparticles, (k) molecular carriers, (l) nanocarriers, (m) nanoscopic multi-variant carriers, (n) nanogels, (o) hybrid nanocarrier systems consisting of components of two or more particulate delivery systems, (p) nanoparticles, (q) peptide-based drug delivery systems, and (r) polymer- or copolymer-based delivery systems.

8. The method of claim 1, wherein the thiol-protected form of the aminothiol is administered in a composition that specifically targets delivery to a selected cell type.

9. The method of claim 2, wherein the thiol-protecting group is not a phosphate moiety.

10. The method of claim 1, wherein the subject is infected with a virus and the aminothiol is administered in an amount effective to exhibit an antiviral effect against the virus.

11. The method of claim 1, wherein the subject is at risk of infection with a virus and the aminothiol is administered in an amount effective to reduce the likelihood that the subject will be infected with the virus.

12. The method of claim 1, wherein the subject is expected to experience a cyto-damaging event and the aminothiol is administered to the subject in an amount sufficient to provide cytoprotection for a period that includes occurrence of the cyto-damaging event.

13. In a method of administering an aminothiol to a subject in need of aminothiol therapy, the improvement comprising administering the aminothiol in a thiol-protected form.

14. The improvement of claim 13, further comprising administering the aminothiol in an intracellular delivery system.

15. A method of treating a viral infection of a subject, the method comprising administering to the subject an anti-virally effective amount of an aminothiol in a thiol-protected form.
